(19) United States
(12) Patent Application Publication
(10) Pub. No.: US 2005/0287077 A1
(43) Pub. Date: Dec. 29, 2005

(54) Process for preparing stable SOL of pharmaceutical ingredients and hydrofluorocarbon

(75) Inventors: Joseph A. Creazzo, Wilmington, DE (US); Sean Mark Dalziel, San Francisco, CA (US); Henricus Jacobus Cornelis Gommeren, Hockessin, DE (US); John Henry Green, Oxford, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(73) Assignee: James E. Shipley, Wilmington, DE (US)

(21) Appl. No.: 11/053,823

(22) Filed: Feb. 9, 2005

Related U.S. Application Data
(60) Provisional application No. 60/543,182, filed on Feb. 10, 2004.

(51) Int. Cl. A61L 9/04; A61K 9/14

(52) U.S. Cl. 424/46

(57) ABSTRACT

The present invention relates to processes for preparing a stable sol of medicament and hydrofluorocarbon, and for preparing medicament delivery devices containing said sol. This invention also relates to a sol composition resulting from said process. This invention further relates to apparatuses for preparing said medicament delivery devices.

### BACKGROUND OF THE INVENTION

Pharmaceutical aerosols have been playing a crucial role in the health and well being of millions of people throughout the world for many years. These products include pressurized metered dose inhalers (pMDIs), dry powder inhalers (DPIs), nebulizers, sublinguals, skin sprays (coolants, anesthetics, etc) and dental sprays. Pulmonary delivery offers an acceptable non-invasive alternative to the needle for systemic administration, for example, for peptides and proteins with poor oral absorption.

Traditionally most pharmaceutical aerosols have been propelled with chlorofluorocarbons (CFCs). Current regulations require pharmaceutical aerosols to be reformulated to contain non-ozone-depleting propellants. In the process of reformulation, there is the opportunity to improve today's pulmonary delivery technology and to create new systems to treat a wide array of infirmities and afflictions.

Alternatives to CFC-propellants must satisfy many other criteria in addition to environmental acceptability. Most importantly, they need to have acceptable toxicity profiles given their use in the delivery of pharmaceutical ingredients. For the sake of patient safety, it is also important that they are nonflammable. Finally, their physical properties must allow workable formulations within the available technology. The two current alternatives to CFC propellants for pharmaceutical aerosols are hydrofluorocarbon (HFC) 134a (also known as hydrofluoroalkane (HFA) 134a or 1,1,1,2-tetrafluoroethane), and HFC-227ea (HFA-227ea or 1,1,1,2,3,3,3-heptafluoropropane).

While HFCs are more environmentally friendly compounds, eliminating chlorine from these molecules has added a significant solvency challenge. As a general rule, replacing chlorine with fluorine reduces solvency. Addressing these reduced solvency characteristics has been a major element of pharmaceutical reformulation programs for replacing CFCs and has amplified the need for improved particle science technology.

Since the mid 1950's, aerosol forms of pharmaceuticals have played an important role in treating respiratory illnesses such as asthma, chronic obstructive pulmonary disease (COPD) and cystic fibrosis; additionally, infectious diseases, prolonged labor and diabetes insipidus are treated and several anesthetics are administered using inhaled pharmaceuticals. More recently, the lung is being considered as a route of delivery for systemic drug delivery, for fast acting treatment (which is important for pain management, diabetes mellitus and others) and for biotech proteins, peptides and gene therapy.

Administration of drugs by the pulmonary route is technically challenging as oral deposition may be high, and variations in inhalation technique may affect the quantity of drug delivered to the lungs. When used to deliver conventional formulations consisting of micronized suspensions it is inefficient. Often formulations deposit less than 15% of the dose in the lungs, while depositing the majority of the dose in the oropharynx. Therefore, there have been considerable efforts to produce more efficient and reproducible aerosol systems through improved drug delivery devices and through better formulations that disperse more readily during inhalation.

With the requirement to reformulate many MDI pharmaceutical products with HFC propellants, a number of technical issues have arisen. Most notably, there is the need to increase the solubility of the active pharmaceutical ingredient in the propellant. The lower solvency power of the pharmaceutical HFC fluids, by comparison with their CFC predecessors, is not easily over come; it is an even tougher technical hill to climb because the group of FDA acceptable solubility enhancing excipients is not so large. Ethanol is the most commonly selected co-solvent. Additionally, water, isopropyl alcohol and polyethylene glycols are commonly used as co-solvents. Ethanol, being a short chain alcohol, will dissolve molecules that have some hydrophobicity, yet its co-solvent properties are not by any means equivalent to the chlorinated alkane CFCs. Consequently, a number of aerosol drug reformulation efforts have been unsuccessful at dissolving the drug into solutions with HFC propellants. So the reverse strategy has been employed—the suspension MDIs.

In a suspension MDI, the drug is relatively insoluble in the propellant and hence drug particles are maintained as a slurry inside the can. While the drug substance itself is usually more chemically stable in the solid-state than in the dissolved-state, there are still quite complex dispersion and device related challenges in formulating a stable microcrystalline suspension MDI. In particular, for suspension MDIs the interfacial chemistry of the suspended drug particles tends to be a dominating factor. Surfactants are required to disperse the particles in their biologically preferred aerodynamic size of 1-5 microns. In many instances, the surfactants that are permissible for inhalation drug formulation are better suited to the interfacial chemistry of CFC products and there is a lengthy road to approval of new surfactants. Hence, the formulator's task is constrained to prototype formulation testing with the standard CFC MDI surfactants. In the case of reformulating combination therapies, the challenges can be twofold, should one drug dissolve in HFC and the other not, since the dissolved species can influence the dispersion. Provided the flocculation rates are not excessively fast, the practice of shaking the MDI immediately prior to dispensing a dose, may deliver sufficient energy to the suspension to temporarily disperse the particles sufficiently. New developments in valves have been brought about to cope with the challenges of metering small precise volumes of slurries, which are more prone to block the orifice of a metering valve and lead to poor dose uniformity.

Conventionally, the active drug particles used in DPIs and suspension MDI particles are prepared by bulk crystallization or freeze drying, followed by fluid energy milling (micronization) to reduce the particle size to around 1-5 microns. Fluid energy milling is a comminution technology that has existed for sixty years and been widely applied in the processing of pigments and pharmaceuticals. In this gas/solids milling process, particles are entrained into a series of jets at high pressure (100-150 psi), where the inter-particle collisions and wall impacts lead to the reduction of particle size. Some compounds cannot substantially be milled down to the inhalable size and thus air classification is required to maximize the fine particle fraction. So while fluid energy milling is widely used as a method of fine particle preparation for inhalation of drugs, it is an area that requires careful attention during product and process development.

Slurry media milling is an important unit operation in various industries for the fine and ultra-fine grinding of minerals, paints, inks, pigments, micro-organisms, food and agricultural products and pharmaceuticals. In these mills, the feed particles are reduced in size between a large number of small grinding media which are usually sand, plastic beads, glass, steel or ceramic beads. As a result of the internally agitated, very small, grinding media and the liquid medium, (aqueous, non-aqueous or a mixture thereof), dispersion products of finer submicron or nanosize particles can be produced, which has not been previously done by conventional mills.

### BRIEF SUMMARY OF THE INVENTION

The present invention relates to a process for preparing a stable sol comprising fine particles of pharmaceutical ingredients (including, but not limited to, medicaments, surfactants, dispersants, solubilizers, binders, diluents, coatings, lubricants, disintegrants, etc.) and liquid hydrofluorocarbon, comprising: milling coarse particles of a pharmaceutical ingredient(s) in a mill in the presence of a hydrofluorocarbon liquid phase and thereby reducing the size of said coarse particles of pharmaceutical ingredient(s) to fine particles of pharmaceutical ingredient(s) and forming a sol comprising fine particles of pharmaceutical ingredient(s) and liquid hydrofluorocarbon. The improvements in sol stability improve the uniformity of both the emitted pharmaceutical ingredient(s) dose and pharmaceutical ingredient(s) delivery to the lung.

### BRIEF DESCRIPTION OF THE DRAWING(S)

FIG. 1: Light Obscuration Data and Model Fits for Two Unmilled Budesonide Formulations

FIG. 2: Light Obscuration Data Demonstrating Stability of Milled Budesonide Formulation

FIGS. 3a AND 3b: Scanning electron micrographs of lactose produced by micronization (jet milling)

FIGS. 4a AND 4b: Scanning electron micrographs of lactose produced by high pressure media milling process

FIG. 5: Obscuration Data for Example 2 (Sample # 214)

FIG. 6: Obscuration Data for Example 2 (Sample # 413)

FIG. 7: Obscuration Data for Example 2 (Sample # 248)

FIG. 8: Obscuration Data for Example 2 (Sample # 415)

FIG. 9: Obscuration Data for Example 5

FIG. 10: Obscuration Data for Example 6

FIG. 11: Obscuration Data for Example 7

FIG. 12: Obscuration Data for Example 8

### DETAILED DESCRIPTION OF THE INVENTION

The present invention is a process for preparing a sol comprising fine particles of pharmaceutical ingredient(s) and liquid hydrofluorocarbon, comprising: a) adding coarse particles of a pharmaceutical ingredient(s) to a mill; b) adding a hydrofluorocarbon to said mill; c) maintaining said mill at a temperature and pressure sufficient to form a hydrofluorocarbon liquid phase; and d) milling said coarse particles of pharmaceutical ingredient(s) in said mill in the presence of said hydrofluorocarbon liquid phase and thereby reducing the size of said coarse particles of pharmaceutical ingredient(s) to fine particles of pharmaceutical ingredient(s) and forming a sol comprising fine particles of pharmaceutical ingredient(s) and liquid hydrofluorocarbon.

The present invention further includes a process for preparing a medical delivery device containing a sol comprising fine particles of pharmaceutical ingredient(s) and liquid hydrofluorocarbon, comprising: a) adding coarse particles of a pharmaceutical ingredient(s) to a mill; b) adding a hydrofluorocarbon to said mill; c) maintaining said mill at a temperature and pressure sufficient to form a hydrofluorocarbon liquid phase; d) milling said coarse particles of pharmaceutical ingredient(s) in said mill in the presence of said hydrofluorocarbon liquid phase and thereby reducing the size of said coarse particles of pharmaceutical ingredient(s) to fine particles of pharmaceutical ingredient(s) and forming a sol comprising fine particles of pharmaceutical ingredient(s) and liquid hydrofluorocarbon; and e) transferring said sol from said mill to a medical delivery device.

The present process may further comprise transferring sol formed in the milling step to a manifold, and then transferring sol from the manifold to one or a plurality of medical delivery devices. The manifold allows for efficacious filling of a medical delivery device with sol formed in the milling step, under temperature and pressure substantially identical to those under which the sol was formed. The manifold comprises any arrangement of piping connecting (a port in) the mill with (a port in) a medical delivery device and allowing for transfer of sol from the mill to the medical delivery device. For simultaneous filling of more than one medical delivery device, the manifold may comprise a main pipe and a plurality of lesser pipes extending therefrom, each lesser pipe connected to a medical delivery device. The manifold may be constructed of materials identical or different from those comprising the mill and/or medical delivery device, for example metal (stainless steel), polymer (PET) and glass.

Sol comprising fine particles of pharmaceutical ingredient(s) and liquid hydrofluorocarbon is formed by milling in a mill, and transferred from the mill to a medical delivery device, such as a MDI. Both milling and transferring steps preferably occur at substantially identical temperature and pressure. When the process comprises milling, transferring sol formed in the milling step to a manifold, and then transferring sol from the manifold to one or a plurality of medical delivery devices, it is preferred that the whole process is carried out at substantially identical temperature and pressure. By substantially identical temperature and pressure means that the temperature and the pressure each change by less than about 15%, preferably less than about 10%, and most preferably by less than about 5%. By retaining the pharmaceutical ingredient(s) particles as a dispersion in liquid hydrofluorocarbon from milling through to device filling and with the concomitant elimination of temperature and pressure variations in the process, the stability of the sol against flocculation and settling is greatly improved. Further, the milling and transferring processes are carried out at a temperature and pressure under which the pharmaceutical ingredient(s) is safe and the hydrofluorocarbon is liquid. The safe temperature to the pharmaceutical ingredient(s) is usually the one under which the pharmaceutical ingredient(s) supplier recommends the pharmaceutical ingredient(s) be stored. Preferred process temperature is ambient temperature.

Pharmaceutical ingredients coarse particles to be milled have an aerodynamic mass mean particle size of greater than about 5 microns. Pharmaceutical ingredients coarse particles may be discrete particles or agglomerates of particles and typically have an aerodynamic mass mean particle size from about 5 to about 100 microns if premilled by another milling process, or may be up to several millimeters if prepared (e.g., crystallized) without premilling.

Pharmaceutical ingredients fine particles resulting from the present milling step have a primary particle diameter range from about 50 to about 5,000 nanometers, which may be confirmed by laser diffraction particle analysis and BET surface area measurement. Upon being expelled from a medical delivery device (e.g., from a metered dose inhaler as an aerosol), the pharmaceutical ingredient(s) fine particles resulting from the present milling step will agglomerate “in flight” as the hydrofluorocarbon propellant evaporates. Hence, agglomeration in flight leads to an aerodynamic behavior of these particles which is consistent with coarser diameter particles than their physical diameter. Aerodynamic mass mean particle size measurement is commonly made using a cascade impactor. Pharmaceutical ingredients fine particles resulting from the present milling step that have agglomerated “in flight” as the hydrofluorocarbon evaporates exhibit an aerodynamic mass mean particle size of about 5 microns or less, preferably from about 1 microns to about 5 microns. Such particles are capable of substantially passing through conducting airways (e.g., trachea, main bronchi, bronchioles) and depositing in the respiratory airways (e.g., terminal bronchioles, respiratory bronchioles, alveolar ducts and alveolar sacs).

A variety of methods may be used to quantify the stability of dispersions. The simplest method is direct visual evaluation where, after agitation, a transparent bottle containing a dispersion is observed. Initially, the contents are opaque and finely dispersed such that the naked eye cannot distinguish any fine structure. If flocculation occurs, first fine, then coarse structure develops which can be visually distinguished. As flocculation continues, clear fluid is apparent between the loose floccules which will eventually separate to a clear solvent-rich layer and a particle rich layer. Whether coarse flocculation occurs or not, the formation of a transparent layer may be observed and its dimensions measured over time. A stable dispersion without flocculation would exhibit no coarse structure and neither a particle-rich phase nor particle-deficient phase would be apparent after standing. Typical phase separation times for dispersions of medicament powders in hydrofluorocarbons are on the order of seconds to minutes. Such a visualization method may be used to determine the relative stability of dispersions.

The present inventors have developed a method, herein referred to as the “obscuration” method, and apparatus useful in statistically designed experiments for optimization of dispersions. In this method, a transparent vessel containing a well-agitated dispersion is placed in a holder which is mounted in a light-proof box. The vessel is illuminated by a light source (e.g., a 60W light bulb) through a 0.75 inch diameter aperture. The light is directed through the upper 90% of the dispersion to a data-logging light meter (e.g., a light meter data logger with PC software, part # 401036, available from Extech Instruments) on the other side of the bottle, and the amount of light passing through this portion of the dispersion is measured over time. This measurement of change in light intensity over time yields the time course of any reflocculation and separation. The light transmittance data, L (lux), is fitted numerically to the time from agitation, t, with Equation 1:

$$L = {B}_{1} + \frac{{B}_{2} - {B}_{1}}{1 + {ⅇ}^{{B}_{3} ( log ( t ) - log ( {B}_{4} ) )}} , \qquad ( Equation 1 )$$

where the coefficients B₁ through to B₄ describe features of the L versus t curve. B₂ is the light reading data (lux) of the fully dispersed state immediately after shaking and B, is the light reading data (lux) of the final state at long time. Theoretically, the final state is reached after the dispersion sits unagitated for infinite time. Practically, the final state is deemed as the state when light reading data no longer changes or changes very slowly over a substantially long time. B₄ is the time for the L to reach halfway between B₁ and B₂. B₃ is the slope of the L versus t curve at B₄. For an easily flocculated and settled dispersion, B₁ is far greater than B₂, and B₄ is relatively short. The desirable, highly stable dispersions of the present invention have B₁ relatively close to B₂, and B₄ is relatively long. For this invention, B₁/B₂ is less than 5 and preferably less than 2. B₄ is at least about 2 minutes, preferably at least one day, more preferably at least one week and most preferably at least two weeks.

Using the obscuration method to differentiate between the performance (relative settling rate) of two dispersions is shown in FIG. 1. In FIG. 1, the L versus t model (Equation 1 as shown above) fits both data sets well and formulation sample #415 performs better than formulation sample #413. Although the levels of reflocculation are similar (B₁s of 4.8 and 4.7 for formulations 415 and 413 respectively), the time for formulation 415 to reflocculate is longer (B₄s of 81 and 56 seconds for formulations 415 and 413 respectively).

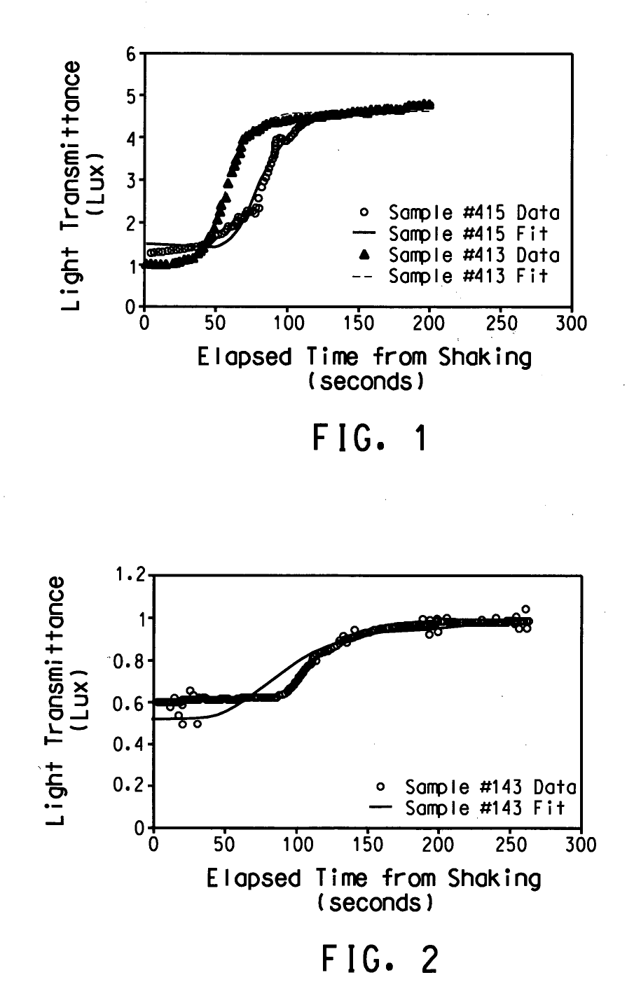

The present invention includes a process for preparing a sol comprising fine particles of pharmaceutical ingredient(s) and liquid hydrofluorocarbon. By sol is meant a stable colloidal dispersion comprising hydrofluorocarbon liquid phase as the dispersion medium, and a colloidal substance, the dispersed phase, comprising pharmaceutical ingredient(s) fine particles, which are distributed throughout the hydrofluorocarbon liquid phase dispersion medium. The present process produces sols of pharmaceutical ingredients in hydrofluorocarbon having surprisingly improved stability over those in the prior art. Improved sol stability is desirable for ensuring dose uniformity and safety of the inhaled dosage form. The sols produced by the present process have obscuration method parameters (Equation 1) as follows: B₁/B₂ is less than 5 and preferably less than 2; B₄ is at least about 2 minutes, preferably at least one day, more preferably at least one week and most preferably at least two weeks. An example of a sol of medicament budesonide formed by the present process using a high pressure media mill is presented in FIG. 2. In FIG. 2, the value of B₁ is close to B₂ (1 and 0.5 respectively), indicating little separation and the value of B₄ is 92 seconds. The L of the dispersion measured in FIG. 2 after two weeks had not substantially changed from the L value at about 300 seconds.

Hydrofluorocarbons of the present invention comprise those suitable for creating and propelling aerosols comprising solid pharmaceutical ingredients and hydrofluorocarbon. Hydrofluorocarbons of the present invention include tetrafluoroethanes (1,1,1,2-tetrafluoroethane (HFC-134a) and 1,1,2,2-tetrafluoroethane (HFC-134)), hexafluoropropanes (1,1,1,3,3,3-hexafluoropropane (HFC-236fa), 1,1,2,2,3,3-hexafluoropropane (HFC-236ca), 1,1,1,2,2,3-hexafluoropropane (HFC-236cb) and 1,1,1,2,3,3-hexafluoropropane (HFC-236ea)) and heptafluoropropanes (1,1,1,2,3,3,3-heptafluoropropane (HFC-227ea) and 1,1,1,2,2,3,3-heptafluoropropane (HFC-227ca)). Preferred are HFC-134a, HFC-227ea and their mixtures.

The ratio of active pharmaceutical ingredient(s) mass to the volume of the hydrofluorocarbon liquid phase is important to the dose delivered from a medical delivery device. Metered dose inhaler metering valves come in different volumes. The ratio of active pharmaceutical ingredient(s) to hydrofluorocarbon liquid phase in micrograms per microliter multiplied by the metering valve volume determines the dispensed dose. Dispensed dose multiplied by the fine particle fraction (i.e., percentage of particles with aerodynamic mass particle size less than 5 microns) equals the respirable dose. Milling pharmaceutical formulations using the present invention can be done across a range of solid loadings for pharmaceutical ingredients in the hydrofluorocarbon liquid phase. The pharmaceutical ingredients can be milled at low loadings, essentially equal to the final product formulation. Alternately, the pharmaceutical ingredients can be milled at higher solids loading, up to 50% solids in the hydrofluorocarbon liquid phase, consistent with the physical configuration of the mill. Formulations milled at higher solids loading can subsequently be diluted with additional hydrofluorocarbon liquid to levels required in the final product. Solids concentrations in final formulations for MDIs are very low. Milling at higher solids loading offers advantages like a higher milling efficiency, higher mill throughput/capacity and reduced contamination from grinding beads.

The milling step of the present process wherein coarse particles of pharmaceutical ingredient(s) are milled in a mill in the presence of a hydrofluorocarbon liquid phase is optionally carried out in the presence of a surfactant. The presence of surfactant increases sol stability. Surfactants of the present invention are chosen from those that do not adversely effect human health when delivered to the pulmonary airways. They may be cationic, amphoteric, nonionic or anionic. The present surfactants may be a halogen-free compound having a molecular weight of about 500 or less or a halogenated compound having a molecular weight of about 1000 or less, and contain a hydrophilic moiety and a hydrophobic moiety. Typical surfactant hydrophobic moiety include aliphatic hydrocarbon groups, fluorocarbon groups, and hydrofluorocarbon groups. Typical surfactant hydrophilic moiety include cationic (e.g., aliphatic ammonium), amphoteric (e.g., amine betaines), nonionic (e.g., oxyalkylene oligomers, sugar alcohols (e.g., sorbitol), mono- and di-saccarides (e.g., sucrose, lactose, maltose)) and anionic (e.g., carboxylate, phosphate, sulfate, sulfonate, sulfosuccinate) groups. Representative surfactants include: stearic acid (CH₃(CH₂)₁₆CO₂H), oleic acid (CH₃(CH₂)₇CH═CH(CH₂)₇CO₂H), sodium lauryl sulfate (CH₃(CH₂)₁₁OSO₃Na), Aerosol OT® (dioctyl sodium sulfosuccinate(Cytec Industries)), Neodol® 25-7 (HO[CH₂CH₂O]₇₋₈(CH₂)₁₂₋₁₅OH (Shell Chemicals)), Span® 80 (sorbitan monooleate (Uniqema)), Ethomeen® C/15 ((C₈₋₁₅ alkyl, primarily C₁₂)N[(CH₂CH₂O)ₘH][CH₂CH₂O)ₙH] (Akzo Nobel)), and Zonyl® FSP (F(CF₂CF₂)₁₋₇CH₂CH₂O)₁₋₂P(O)(ONH₄)₁₋₂ (DuPont)). A preferred surfactant is sodium lauryl sulfate (CH₃(CH₂)₁₁OSO₃Na).

The amount of surfactant used in the present milling process may be from about 0 weight percent up to the solubility limit of said surfactant in a particular formulation of medicament/hydrofluorocarbon/surfactant/optional dispersant, preferably from about 0 weight percent to about 0.5 weight percent, based on the total weight of hydrofluorocarbon, surfactant, medicament and optional dispersant.

The milling step of the present process wherein coarse particles of medicament are milled in a mill in the presence of a hydrofluorocarbon liquid phase is optionally carried out in the presence of a dispersant. The presence of dispersant increases sol stability. Dispersants of the present invention are chosen from those that do not adversely effect human health when delivered to the pulmonary airways. They may be cationic, amphoteric, nonionic or anionic. The present dispersants may have a molecular weight of about 500 or greater and contain a hydrophilic moiety and a hydrophobic moiety. Typical dispersant hydrophobic moiety include aliphatic hydrocarbon groups, fluorocarbon groups and hydrofluorocarbon groups. Typical dispersant hydrophilic moiety include cationic (e.g., aliphatic ammonium), amphoteric (e.g., amine betaines), nonionic (e.g., oxyalkylene oligomers, sugar alcohols (e.g., sorbitol), polysorbates, polysaccarides) and anionic (e.g., carboxylate, phosphate, sulfate, sulfonate, sulfosuccinate) groups. Representative dispersants include: phospholipids (e.g., soy lecithin), polysaccharides (e.g., starch, glycogen, agar, carrageenan), polysorbate 80, Span® 85 (sorbitan trioleate (Uniqema)), Pluronics 25R4 and Pluronics P104.

The amount of dispersant used in the present milling process may be from about 0 weight percent up to the solubility limit of said dispersant in a particular formulation of medicament/hydrofluorocarbon/optional surfactant/dispersant, preferably from about 0 weight percent to about 0.5 weight percent, based on the total weight of hydrofluorocarbon, optional surfactant, medicament and dispersant.

The milling step of the present process wherein coarse particles of pharmaceutical ingredient(s) are milled in a mill in the presence of a hydrofluorocarbon liquid phase is optionally carried out in the presence of a cosolvent. The presence of a cosolvent improves the performance of non-fluorinated surfactants. Cosolvents of the present invention are chosen from those that do not adversely effect human health when delivered to the pulmonary airways. Representative cosolvents include water, ethanol, isopropyl alcohol, polyethylene glycol, propylene glycol and dipropylene glycol.

Mills of the present invention are generally any device or method that achieves reduction in the size of coarse particles of pharmaceutical ingredient(s) through a grinding process, optionally utilizing grinding media. The present milling process can be any slurry grinding process that uses an attritor, a tumbling ball mill, a vibratory ball mill, a planetary ball mill, a horizontal media mill, a vertical media mill, an annular media mill, a rotor-stator or a high pressure media mill. Preferred of the mills is a high pressure media mill as disclosed in U.S. patent application Ser. No. 10/476,312, incorporated herein by reference. The present milling step comprises a liquid milling process also called slurry milling, wherein a liquid hydrofluorocarbon is used as the carrier fluid.

The milling step of the present invention optionally uses grinding media, which is added to the mill prior to milling. Grinding media is generally known to those of ordinary skill in this field and is generally comprised of any material of greater hardness and rigidity than the medicament to be ground. The grinding media can be comprised of almost any hard, tough material including, for example, nylon and polymeric resins, metals, and a range of naturally occurring substances, such as sand, silica, or chitin obtained from crab shells. Preferably, grinding media of the present invention is comprised of a tough resilient material having a low rate of attrition, and therefore a low incidence of contamination of the medicament fine particles with attrited media pieces. Further, grinding media may either consist entirely of a single material that is tough and resilient, or in the alternative, be comprised of more than one material, i.e., comprise a core portion having a coating of tough resilient material adhered thereon. Additionally, the grinding media may be comprised of mixtures of any materials that are suitable for grinding. The polymeric resins suitable for use herein as grinding media are chemically and physically inert, preferably substantially free of metals, solvents and monomers, and of sufficient hardness and friability to avoid being chipped and crushed during grinding. Suitable polymeric resins include, but are not limited to, crosslinked polystyrenes, such as polystyrene crosslinked with divinylbenzene, styrene copolymers, polycarbonates, polyacetals, such as Delrin®, vinyl chloride polymers and copolymers, polyurethanes, polyamides, poly(tetrafluoroethylenes), e.g., Teflon®, and other fluoropolymers, high density polyethylenes, polypropylenes, cellulose ethers and esters such as cellulose acetate, polyhydroxymethacrylate, polyhydroxyethylacrylate, silicone containing polymers such as polysiloxanes and the like. Biodegradable polymeric resins are also suitable for use herein as grinding media. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). In the case of biodegradable polymers, media contaminants can be advantageously metabolized in vivo to biologically acceptable products that can be eliminated from the body. Additional grinding media materials include digestible ingredients having “GRAS” (generally recognized as safe) status. For instance, starch based materials or other carbohydrates, protein based materials, and salt based materials. Any size of grinding media suitable to achieve the desired particle size can be utilized. However, in many applications the preferred size range of the grinding media will be in the 15 mm to 20 micron range for continuous media milling with media retention in the mill. For batch media milling (in attritors) or circulation milling in which slurry and grinding media are circulated, smaller nonspherical grinding media can be often utilized.

In the instance where a pharmaceutical ingredient(s) is milled to form a pharmaceutical ingredient(s) and hydrofluorocarbon sol in the presence of grinding media, and said sol is transferred directly to a medical delivery device (e.g., a metered dose inhaler (MDI)), the grinding media is preferably removed before said transferring by procedures know to those skilled in this field. For example, the bottom of a mill grinding chamber may contain a grinding media retention screen. The grit of the screen is sufficiently small to retain the grinding media and allow the sol to pass through substantially free of grinding media.

In the instance of air or water sensitive pharmaceutical ingredient(s), milling of the present invention may involve evacuating gases from the mill prior to said adding of a hydrofluorocarbon to the mill, and/or purging the mill with an inert gas prior to said adding of a hydrofluorocarbon to said mill.

Pharmaceutical ingredients of the present invention are friable, crystalline or amorphous, solids that are poorly soluble in hydrofluorocarbon. By “poorly soluble”, is meant that the pharmaceutical ingredients has a solubility in the hydrofluorocarbon of less than about 10 mg/ml, and in most instances less than about 1 mg/ml, at room temperature. However, pharmaceutical ingredients that are not poorly soluble can still be milled by utilizing hydrofluorocarbon that is saturated with a pharmaceutical ingredient. Further, the present invention may be used in the milling, formulation and device filling of combination therapies. In such a case, at least one of the therapeutic or excipient agents need to be insoluble or saturated in the hydrofluorocarbon. Thus, the present invention has application where one or more of the active or inactive ingredients is not completely dissolved into the hydrofluorocarbon under the thermodynamic conditions of use. Medicaments of the present invention exist in the classes of anti-asthmatics, antibiotics, anticholinergics, anti-inflammatories, beta-agonists, bronchospasmolytic drugs, bronchodilators, corticosteriods, decongestants, diagnostics, expectorants, hormones, hormone replacement therapy drugs, immunosuppressants, mucolytics, pain relievers, proteins, peptides, vaccines, nucleic acids, recombinant proteins and enzymes. Medicaments of the present invention include the inhaled locally acting drugs: albuterol, beclomethasone dipropionate, bitolterol, budesonide, cromolyn sodium, dexamethasone, dornase alfa, rDNAase, ephedrine, epinephrine, ethylnorepinephrine, fenoterol, flunisolide, fluticasone propionate, formoterol, growth hormone, hydrocortisone, insulin, ipratropium bromide, isoetharine, isoproteranol levalbuterol hydrochloride, metaproterenol, morphine, nedrocromil sodium, pirbuterol, salbutamol, salmeterol, terbutaline, tiotropium bromide, and triamcinolone acetonide.

### EXAMPLES

### Example 1

### Milling and Dispersion of Lactose Monohydrate with Surfactant 1N HFC-134a Using Pressurized Media Mill

A pressurized, high speed, stirred media mill (as disclosed in U.S. patent application Ser. No. 10/476,312) was charged with 1,700 g of grinding beads (SEPR® 0.8/1.0 mm), 3.47 g of lactose monohydrate, and 3.46 g of surfactant Span® 85 (sorbitan trioleate). The mill was charged with 695 g of HFC 134a. The mill agitator speed was set at 1,776 rpm. The milling process was run for 15 minutes at a temperature of 25° C. and pressure of 20 bars.

The sol (pressurized slurry) of milled lactose particles and surfactant in HFC-134a was discharged into a glass collection bottle. The sol was observed to be extremely stable; no significant flocculation or creaming was observed after 3 weeks storage without agitation.

After venting off the HFC-134a propellant, active particles were collected and analyzed for particle size (by Malvern Mastersizer) and specific surface area (by BET). Results are presented in Table 1. The median particle size of the sample measured by the Malvern Mastersizer is 4.3 microns. The BET specific surface area is 5.8 m²/gram indicating that the particles are actually agglomerates, consisting of sub micron particles.

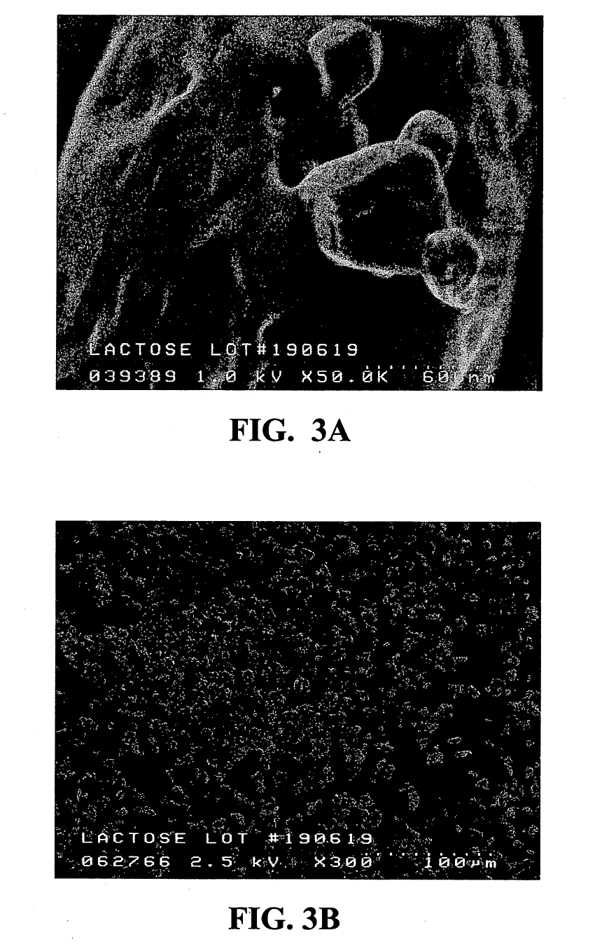

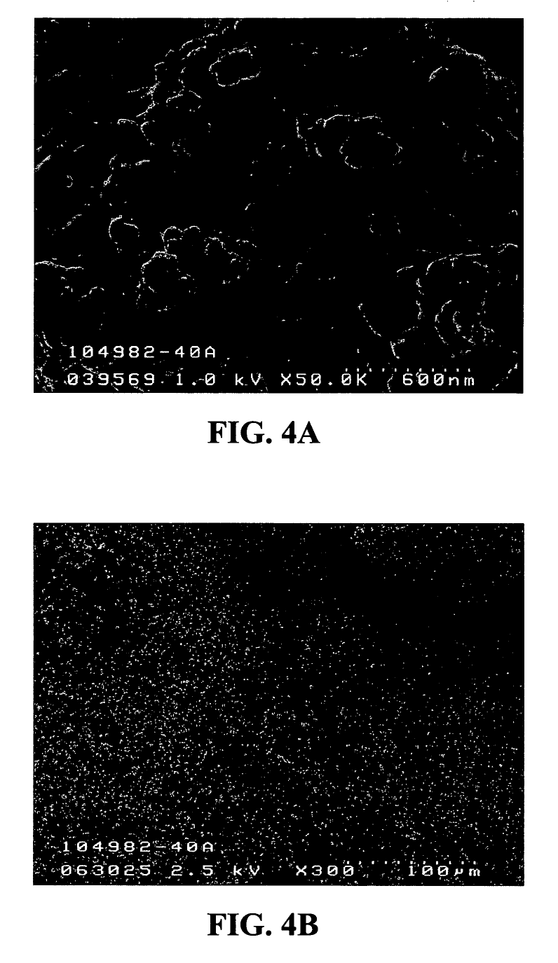

FIGS. 3a, 3b, 4a and 4b show scanning electron micrographs of jet milled lactose particles (3a and 3b) and the high pressure media milled lactose (4a and 4b) produced in this example.

TABLE 1
Particle size and surface area of lactose milled in HFC-134a.

| | ×10 | ×50 | ×90 | BET |
|---|---|---|---|---|
| | [micron] | [micron] | [micron] | [m²/g] |
| Lactose/HFC-134a | 1.06 | 4.30 | 553 | 5.8 |

### Comparative Example 2

### Unmilled Budesonide Formulations

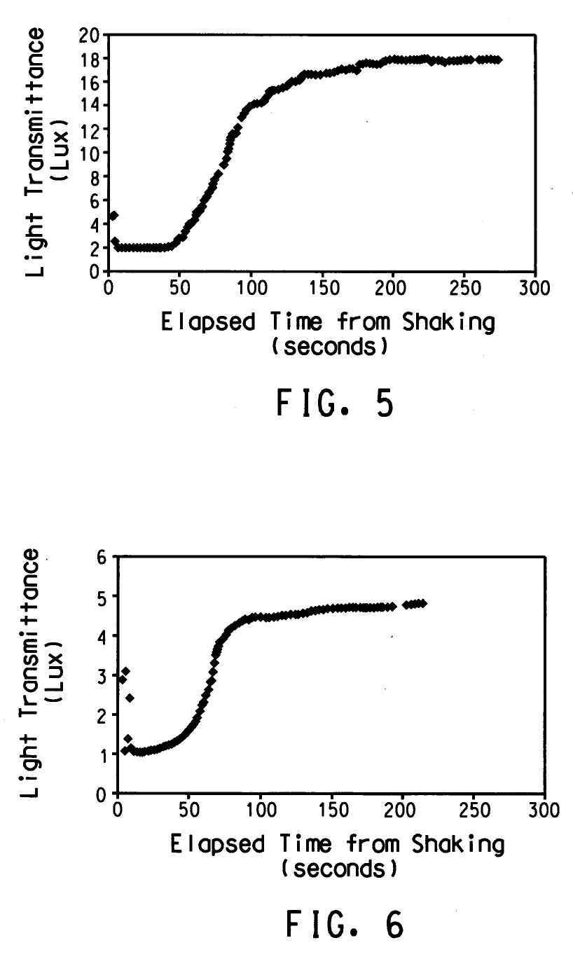

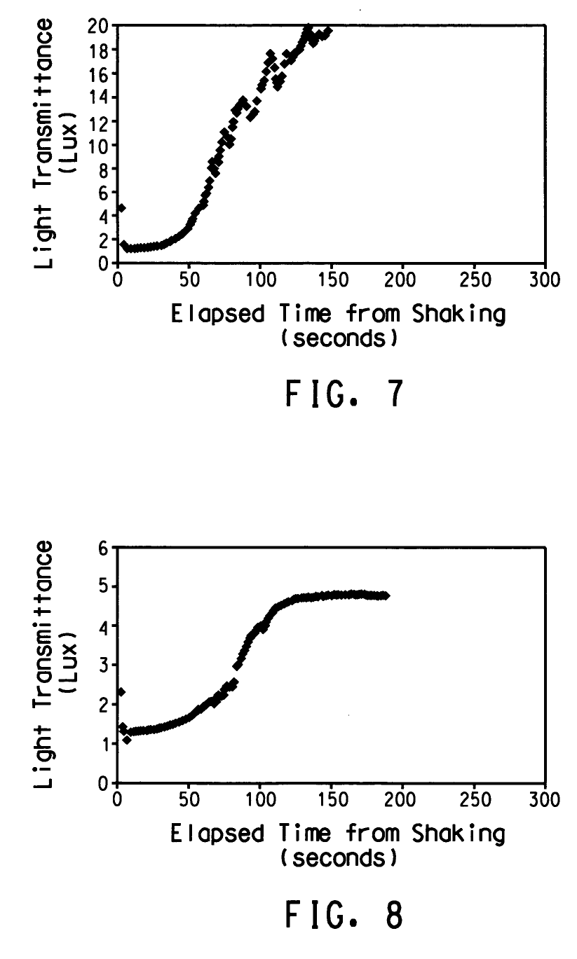

Formulations using micronized budesonide were prepared in glass bottles by mixing budesonide with propellant with and without other additives. The mixtures were shaken and allowed to settle. Stability of the mixtures was measured using the obscuration method describe previously. The following comparative examples show how quickly the unmilled mixtures separate.
Sample #214: 0.5 weight % budesonide in HFC-134a propellant only (Obscuration data in FIG. 5)
Sample #413: 0.5 weight % budesonide in HFC-227ea propellant only (Obscuration data in FIG. 6)
Sample #248: 0.5 weight % budesonide and 0.5 weight % sodium lauryl sulfate surfactant in HFC-134a propellant (Obscuration data in FIG. 7)
Sample #415: 0.5 weight % budesonide and 0.5 weight % dipropylene glycol co-solvent in a 50/50, by weight, mixture of HFC-134a/HFC-227ea (Obscuration data in FIG. 8)

### Example 3

### Milling of Budesonide with Surfactant in Pressurized Propellant HFC-134a

A pressurized high speed stirred media mill (as disclosed in U.S. patent application Ser. No. 10/476,312) was charged with 1,700 g of grinding beads (SEPR® 0.8/1.0 mm), 3.47 g of budesonide, and 3.46 g of surfactant SLS (sodium lauryl sulfate (CH₃(CH₂)₁₁OSO₃Na)). The mill was charged with 693 g of the propellant HFC-134a. The mill agitator speed was 1,776 rpm. The milling process was run for 60 minutes at a temperature or 25° C. and pressure of 20 bars.

The sol (pressurized slurry) of HFC-134a, milled budesonide particles and surfactant was discharged into a glass bottle. No significant flocculation or creaming was observed by visual observation after 3 weeks storage without agitation.

### Example 4

### Cold Fill of Micronized Budesonide with a Mixture of Hydrofluorocarbons

57 milligrams of micronized budesonide from Spectrum (lot# RB0362) and 57 milligrams of surfactant SLS (sodium lauryl sulfate (CH₃(CH₂)₁₁OSO₃Na) were added to the MDI canister. The MDI canister was charged with 11.5 g of a mixture of HFC-134a and HFC-227ea resulting in the following mixture:

| Compound | Concentration [wt %] |
|---|---|
| SLS | 0.50 |
| Budesonide | 0.50 |
| HFC-134 | 64.9 |
| HFC-227ea | 34.1 |

The fine particle fraction (FPF), median mass aerodynamic diameter (MMAD) and throat deposition (>10%) were determined using the Andersen Cascade Impact (ACI) tester, following the procedures for 5 metered dose inhalers as described in USP 26, Chapter 601 Aerosols, metered dose inhalers and dry powder inhalers, page 2105-2123. An MDI mouthpiece with 0.7 mm orifice diameter was used.

TABLE 2
ACI data for Example 4 (0.7 mm mouthpiece orifice)

| | | |
|---|---|---|
| Median Mass Diameter (MMAD) | 8.925 | micron |
| Fine Particle Fraction (FPF) | 3.98 | % |
| Throat deposition (>10 micron) | 69.92 | % |

### Example 5

### Milling of Budesonide with Surfactant and Dispersant with a Mixture of Hydrofluorocarbons

A high pressure media mill (as disclosed in US patent application No. 10/476,312) was charged with 1,700 g of grinding beads (SEPR® 0.8/1.0 mm), 3.47 g of budesonide, 3.3 g of surfactant SLS (sodium lauryl sulfate (CH₃(CH₂)₁₁OSO₃Na) and 0.97 g of dispersant soy lecithin. The mill was charged with 693 g of a mixture of HFC-134a and HFC-227ea resulting in the following mixture:

| Compound | Concentration [wt %] |
|---|---|
| SLS | 0.47 |
| soy lecithin | 0.14 |
| budesonide | 0.49 |
| HFC-134 | 64.9 |
| HFC-227ea | 34.0 |

The mill agitator speed was 1,776 rpm. The milling process was run for 15 minutes at a temperature of 25° C. and pressure of 20 bars. The sol (pressurized slurry) of hydrofluorocarbons, milled budesonide particles, surfactant and dispersant was discharged into a cylinder. MDI canisters and glass bottles were filled directly from this cylinder. No significant flocculation or creaming was observed by visual observation after 3 weeks storage without agitation. The performances of the MDIs were characterized with an ACI after 8 months using the procedures as described in USP 26, chapter 601, Aerosols, pages 2105-2123. Mouthpieces with a 0.7 mm orifice and a 0.3 mm mouthpiece were used. Results are listed in tables 3a and 3b. The use of a smaller mouthpiece with the produced sol formulation resulted in a significantly improved fine particle fraction and lower throat deposition.

TABLE 3a
ACI data for Example 5 (0.7 mm mouthpiece orifice)

| | | |
|---|---|---|
| Median Mass Diameter (MMAD) | 5.51 | micron |
| Fine Particle Fraction (FPF) | 18.41 | % |
| Throat deposition (>10 micron) | 54.51 | % |

TABLE 3b
ACI data for Example 5 (0.3 mm mouthpiece orifice)

| | | |
|---|---|---|
| Median Mass Diameter (MMAD) | 4.16 | micron |
| Fine Particle Fraction (FPF) | 36.31 | % |
| Throat deposition (>10 micron) | 37.27 | % |

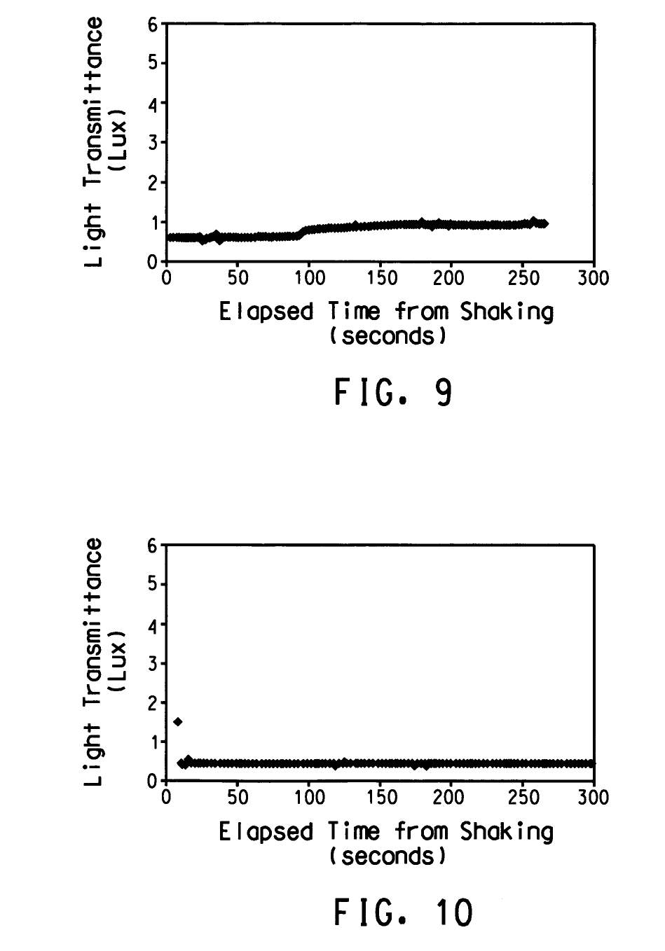

Obscuration data in FIG. 9.

### Example 6

### Milling of Budesonide in Mixture of Hydrofluorocarbons

A high pressure media mill (as disclosed in U.S. patent application Ser. No. 10/476,312) was charged with 1,700 g of grinding beads (SEPR® 0.8/1.0 mm), and 3.5 g of budesonide. The mill was charged with 695 g of a mixture of HFC-134a and HFC-227ea resulting in the following mixture:

| Compound | Concentration [wt %] |
|---|---|
| Budesonide | 0.5 |
| HFC-134 | 64.9 |
| HFC-227ea | 34.6 |

The mill agitator speed was 1,775 rpm. The milling process was run for 15 minutes at a temperature of 25° C. and pressure of 20 bars. The sol (pressurized slurry) of hydrofluorocarbons, milled budesonide particles, surfactant and dispersant was discharged through a coil submerged in a cooling bath containing a mixture of dry ice and acetone. The MDI canisters were filled directly with the supercooled/liquefied sol.

The fine particle fraction (FPF), median mass aerodynamic diameter (MMAD) and throat deposition (>10%) were determined using the Andersen Cascade Impact (ACI) tester, following the procedures for metered dose inhalers as described in USP 26, Chapter 601 Aerosols, metered dose inhalers and dry powder inhalers, page 2105-2123. An MDI mouthpiece with 0.7 mm orifice diameter was used.

TABLE 4
ACI data for Example 6 (0.7 mm mouthpiece orifice)

| | | |
|---|---|---|
| Median Mass Diameter (MMAD) | 3.75 | micron |
| Fine Particle Fraction (FPF) | 30.24 | % |
| Throat deposition (>10 micron) | 50.56 | % |

Obscuration data in FIG. 10.

### Example 7

### Milling of Budesonide with Surfactant in Mixture of Hydrofluorocarbons

A high pressure media mill (as disclosed in U.S. patent application Ser. No. 10/476,312) was charged with 1,700 g of grinding beads (SEPR® 0.8/1.0 mm), 3.47 g of budesonide, 3.47 g of surfactant SLS (sodium lauryl sulfate (CH₃(CH₂)₁₁OSO₃Na). The mill was charged with 695 g of a mixture of HFC-134a and HFC-227ea resulting in the following mixture:

| Compound | Concentration [wt %] |
|---|---|
| SLS | 0.5 |
| Budesonide | 0.5 |
| HFC-134 | 65.0 |
| HFC-227ea | 34.0 |

The mill agitator speed was 1,775 rpm. The milling process was run for 15 minutes at a temperature of 25° C. and pressure of 20 bars. The sol (pressurized slurry) of hydrofluorocarbons, milled budesonide particles, surfactant and dispersant was discharged through a coil submerged in a cooling bath containing a mixture of dry ice and acetone. The MDI canisters were filled directly with the supercooled/liquified sol.

The fine particle fraction (FPF), median mass aerodynamic diameter (MMAD) and throat deposition (>10%) were determined using the Andersen Cascade Impact (ACI) tester, following the procedures for metered dose inhalers as described in USP 26, Chapter 601 Aerosols, metered dose inhalers and dry powder inhalers, page 2105-2123. An MDI mouthpiece with 0.7 mm orifice diameter was used.

TABLE 5
ACI data for Example 7 (0.7 mm mouthpiece orifice)

| | | |
|---|---|---|
| Median Mass Diameter (MMAD) | 5.11 | micron |
| Fine Particle Fraction (FPF) | 18.10 | % |
| Throat deposition (>10 micron) | 58.60 | % |

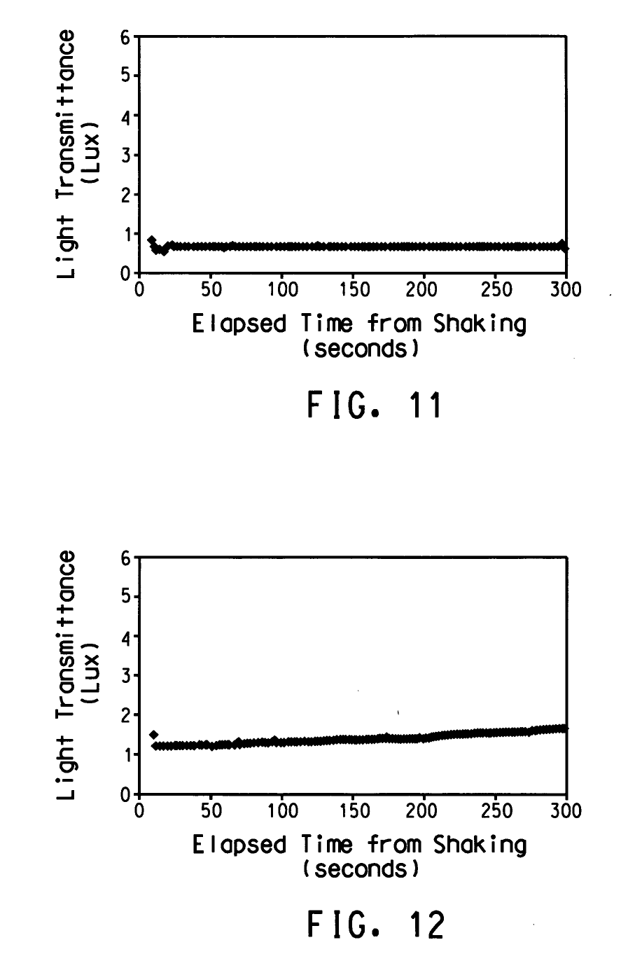

Obscuration data in FIG. 11.

### Example 8

### Millling of Concentrated Budesonide Formulations in HFC

A pressurized high speed stirred media mill (as disclosed in U.S. patent application Ser. No. 10/476,312) was charged with 1,700 g of grinding beads (SEPR® 0.8/1.0 mm), budesonide, sodium lauryl sulfate surfactant, and a blend of HFC-134a/HFC-227ea propellants. The formulation was milled at 2.5WT % solids concentration. The mill agitator speed was 1,776 rpm. The milling process was run for 60 minutes at a temperature or 25° C. and pressure of 20 bars. When milling was complete, the high solids mixture was charged into a mixing vessel and diluted to 0.5 wt % budesonide with neat mixture of HFC-134a/HFC-227ea propellants. The sol (pressurized slurry) was directly loaded to MDI containers via a cold-filling apparatus added to the discharge line on the mill and an MDI aerosol valve was crimped onto the canister as it was loaded. This technique simulated direct filling of MDIs from the milling process.

The fine particle fraction (FPF), median mass aerodynamic diameter (MMAD) and throat deposition (>10%) were determined using the Andersen Cascade Impact (ACI) tester, following the procedures for metered dose inhalers as described in USP 26, Chapter 601 Aerosols, metered dose inhalers and dry powder inhalers, page 2105-2123. An MDI mouthpiece with 0.7 mm orifice diameter was used.

TABLE 6
ACI data for Example 8 (0.7 mm mouthpiece orifice)
Milling at 2.5 wt % solids followed by dilution to 0.5 wt % solids.

| | | |
|---|---|---|
| Median Mass Diameter (MMAD) | 3.125 | micron |
| Fine Particle Fraction (FPF) | 35.76 | % |
| Throat deposition (>10 micron) | 52.87 | % |

Obscuration data in FIG. 12.

1. A process for preparing a sol comprising fine particles of pharmaceutical ingredients and liquid hydrofluorocarbon, comprising:
a) adding coarse particles of pharmaceutical ingredients to a mill;
b) adding a hydrofluorocarbon to said mill;
c) maintaining said mill at a temperature and pressure sufficient to form a hydrofluorocarbon liquid phase; and
d) milling said coarse particles of pharmaceutical ingredients in said mill in the presence of said hydrofluorocarbon liquid phase and thereby reducing the size of said coarse particles of pharmaceutical ingredients to fine particles of pharmaceutical ingredients and forming a sol comprising fine particles of pharmaceutical ingredients and liquid hydrofluorocarbon.

2. A process for preparing a medical delivery device containing a sol comprising fine particles of pharmaceutical ingredients and liquid hydrofluorocarbon, comprising:
a) adding coarse particles of a pharmaceutical ingredients to a mill;
b) adding a hydrofluorocarbon to said mill;
c) maintaining said mill at a temperature and pressure sufficient to form a hydrofluorocarbon liquid phase;
d) milling said coarse particles of pharmaceutical ingredients in said mill in the presence of said hydrofluorocarbon liquid phase and thereby reducing the size of said coarse particles of pharmaceutical ingredients to fine particles of pharmaceutical ingredients and forming a sol comprising fine particles of pharmaceutical ingredients and liquid hydrofluorocarbon; and
e) transferring said sol from said mill to a medical delivery device.

3. The process of claims 2, further comprising transferring said sol formed in said milling step to a manifold, and then transferring said sol from said manifold to said medical delivery device.

4. The process of claims 2 or 3, wherein said milling and each said transferring is performed at substantially the same temperature.

5. The process of claims 2 or 3, wherein said milling and each said transferring is performed at substantially the same pressure.

6. The process of claims 1, 2, or 3, wherein said milling is carried out at ambient temperature.

7. The process of claims 1 or 2, wherein said pharmaceutical ingredients coarse particles have an aerodynamic mass mean particle size of greater than about 5 microns.

8. The process of claims 1 or 2, wherein said pharmaceutical ingredients fine particles have at least 40% of particles with an aerodynamic mass mean particle size of about 5 microns or less.

9. The process of claims 1 or 2, wherein said pharmaceutical ingredients fine particles have a primary particle diameter range from about 50 to about 5,000 nanometers.

10. The process of claims 1 or 2, wherein said sol of pharmaceutical ingredients fine particles in liquid hydrofluorocarbon has obscuration method value B₁/B₂ less than 2, and B₄ greater than at least one week.

11. The process of claims 1 or 2, wherein said hydrofluorocarbon comprises at least one hydrofluorocarbon selected from the group consisting of tetrafluoroethanes, hexafluoropropanes and heptafluoropropanes.

12. The process of claims 1 or 2, wherein said hydrofluorocarbon comprises a mixture of HFC-134a and HFC-227ea.

13. The process of claims 1 or 2 wherein said milling is further carried out in the presence of a surfactant.

14. The process of claim 13 wherein said surfactant is at least one surfactant selected from the group consisting of: stearic acid (CH₃(CH₂)₁₆CO₂H), oleic acid (CH₃(CH₂)₇CH═CH(CH₂)₇CO₂H), sodium lauryl sulfate (CH₃(CH₂)₁₁OSO₃Na), Aerosol OT® (dioctyl sodium sulfosuccinate), Neodol® 25-7 (HO[CH₂CH₂O]₇₋₈(CH₂)₁₂₋₁₅OH), Span® 80 (sorbitan monooleate), Ethomeen® C/15 ((C₈₋₁₅ alkyl, primarily C₁₂)N[(CH₂CH₂O)ₘH][CH₂CH₂O)ₙH]), and Zonyl® FSP (F(CF₂CF₂)₁₋₇CH₂CH₂O)₁₋₂P(O)(ONH₄)₁₋₂).

15. The process of claims 1 or 2 wherein said milling is further carried out in the presence of a dispersant.

16. The process of claim 15 wherein said dispersant is at least one dispersant selected from the group consisting of soy lecithin, starch, glycogen, agar, carrageenan, polysorbate 80, Span® 85 (sorbitan trioleate), Pluronics 25R4 and Pluronics P104.

17. The process of claims 1 or 2 wherein said milling is further carried out in the presence of a cosolvent.

18. The process of claim 17 wherein said cosolvent is at least one cosolvent selected from the group consisting of: water, ethanol, isopropyl alcohol, polyethylene glycol, propylene glycol and dipropylene glycol.

19. The process of claims 1 or 2, wherein said mill is selected from the group consisting of: attritors, tumbling ball mills, vibratory ball mills, planetary ball mills, horizontal media mills, vertical media mills, annular media mills, high pressure media mills and rotor-stators.

20. The process of claims 1 or 2 wherein said mill is a high pressure media mill.

21. The process of claim 20, further comprising adding grinding media to said mill prior to said milling.

22. The process of claims 1 or 2, further comprising evacuating gases from said mill prior to said adding of a hydrofluorocarbon to said mill.

23. The process of claims 1 or 2, further comprising purging said mill with an inert gas prior to said adding of a hydrofluorocarbon to said mill.

24. The process of claims 1 or 2, wherein said pharmaceutical ingredient is at least one medicament selected from the group consisting of: anti-asthmatics, antibiotics, anti-inflammatories, bronchospasmolytic drugs, bronchodilators, corticosteriods, decongestants, diagnostics, expectorants, hormones, hormone replacement therapy drugs, immunosuppressants, mucolytics, pain relievers, proteins, peptides, vaccines, nucleic acids, recombinant proteins and enzymes.

25. The process of claims 1 or 2, wherein said pharmaceutical ingredients is at least one medicament selected from the group consisting of: budesonide, ipratropium bromide, albuterol, salbutamol, salmeterol xinafoate, levalbuterol hydrochloride, flunisolide, metaproterenol, formoterol fumarate, pirbuterol acetate, epinephrine, beclomethasone dipropionate, fenoterol, tiotropium bromide, fluticasone propionate, triamcinolone acetonide, morphine, growth hormone, dornase alfa, rDNAase and insulin.

26. An apparatus for preparing a medical delivery device containing a sol comprising fine particles of pharmaceutical ingredients and liquid hydrofluorocarbon, comprising,
a) a mill comprising i) a milling chamber capable of holding material at elevated pressures, ii) stirring means, and iii) a port;
b) a manifold; and
c) a medical delivery device containing a port,
wherein said manifold connects said port in said mill to said port in said medical delivery device.

27. The apparatus of claim 26 wherein said milling chamber is capable of holding material at pressures up to about 400 psig.

28. The apparatus of claim 26 wherein said milling chamber is capable of holding material at pressures up to about 1,000 psig.

29. A sol, comprising:
a) fine particles of a pharmaceutical ingredient or ingredients;
b) a surfactant;
c) optionally, a dispersant;
d) optionally, a cosolvent; and
e) a liquid hydrofluorocarbon, made by the process comprising:
a) adding coarse particles of pharmaceutical ingredient(s) to a mill;
b) adding a hydrofluorocarbon to said mill;
c) maintaining said mill at a temperature and pressure sufficient to form a hydrofluorocarbon liquid phase; and
d) milling said coarse particles of pharmaceutical ingredient(s) in said mill in the presence of said hydrofluorocarbon liquid phase, said surfactant, optionally, said dispersant, and optionally, said cosolvent, and thereby reducing the size of said coarse particles of pharmaceutical ingredient(s) to fine particles of pharmaceutical ingredient(s) and forming said sol.
